(19) United States
(12) Patent Application Publication
(10) Pub. No.: US 2018/0237424 A1
(43) Pub. Date: Aug. 23, 2018

(54) FGFR3 ANTAGONISTS

(71) Applicants: INSERM (INSTITUT NATIONAL DE LA SANTE ET DE LA RECHERCHE MEDICALE), Paris (FR); UNIVERSITE PARIS DESCARTES, Paris (FR); FONDATION IMAGINE, Paris (FR); ASSISTANCE PUBLIQUE-HOPITAUX DE PARIS (APHP), Paris (FR); CENTRE NATIONAL DE LA RECHERCHE SCIENTIFIQUE (CNRS), Paris (FR)

(72) Inventors: Laurence LEGEAI-MALLET, Paris (FR); Ludovic MARTIN, Paris (FR); Patricia BUSCA, Paris (FR); Laurent CORRE, Paris (FR)

(21) Appl. No.: 15/554,749

(22) PCT Filed: Mar. 2, 2016
(86) PCT No.: PCT/EP2016/054383

§ 371 Date: Aug. 31, 2017

(30) Foreign Application Priority Data
Mar. 3, 2015 (EP) 15305321.0

(51) Int. Cl. C07D 413/04; C07D 413/14

(52) U.S. Cl. CPC C07D 413/04; C07D 413/14

(57) ABSTRACT

The invention pertains to novel FG-FR3antagonists of general formula (I), The compounds are useful for the treatments and prevention of achondroplasia and cancer.

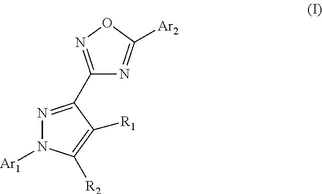

### FIELD OF THE INVENTION

The present invention relates to new oxadiazole compounds which are antagonists of the fibroblast growth factor receptor 3 (FGFR3) for use in the treatment or prevention of FGFR3-related skeletal diseases and cancer.

### BACKGROUND OF THE INVENTION

Skeletal development in humans is regulated by numerous growth factors. Among them Fibroblast Growth Factor Receptor 3 (FGFR3) has been described as both a negative and a positive regulator of endochondral ossification.

The FGFR3 gene, which is located on the distal short arm of chromosome 4, encodes a 806 amino acid protein precursor (fibroblast growth factor receptor 3 isoform 1 precursor; SEQ ID NO: 1).

The FGFR3 protein belongs to the receptor-tyrosine kinase family. This family comprises receptors FGFR1, FGFR2, FGFR3 and FGFR4 that respond to fibroblast growth factor (FGF) ligands. These structurally related proteins exhibit an extracellular domain composed of three immunoglobin-like domains which form the ligand-binding domain, an acid box, a single transmembrane domain and an intracellular split tyrosine kinase domain. Although to date the physiological ligand(s) for FGFR3 is (are) not known, like other FGFRs, it is activated by FGF ligands. Binding of one of the 22 FGFs induces receptor dimerization and autophosphorylation of tyrosine residues in the cytoplasmic domain. The phosphorylated tyrosine residues are required for activation of the signaling pathways. The most relevant tyrosines are Y648, Y647, located in the activation loop.

Several signaling pathways have been described downstream of FGFR3 activation, including the ERK and p38 MAP kinase pathways (Legeai-Mallet et al., J Biol Chem, 273: 13007-13014, 1998; Murakami et al., Genes Dev, 18: 290-305, 2004; Matsushita et al., Hum Mol Genet, 18: 227-240, 2009; Krejci et al., J Cell Sci, 121: 272-281, 2008) and the signal transducer and activation of transcription (STAT) pathway (Su, W. C. et al., Nature, 386: 288-292, 1997; Legeai-Mallet et al., Bone, 34: 26-3, 2004; Li, C. et al., Hum Mol Genet, 8: 35-44, 1999). Others pathways in endochondral bone growth have been identified such as the phosphoinositide 3 kinase-AKT (Ulici, V. et al., Bone, 45: 1133-1145, 2009) and protein kinase C pathways. The degradation of mutant receptors is disturbed as demonstrated by higher levels of FGFR3 mutant receptors at the cell surface (Monsonego-Ornan et al., Mol Cell Biol, 20: 516-522, 2000; Monsonego-Ornan et al., FEBS Lett, 528: 83-89, 2002; Delezoide et al., Hum Mol Genet, 6: 1899-1906, 1997), and disruption of c-Cbl-mediated ubiquitination (Cho, J. Y. et al., Proc Natl Acad Sci USA, 101: 609-614, 2004). FGFR3 mutations disrupt the formation of glycosylated isoforms of the receptor and impede its trafficking (Gibbs et al., Biochim Biophys Acta, 1773: 502-512, 2007; Bonaventure et al., FEBS J, 274: 3078-3093, 2007).

While long bone development involves endochondral ossification, craniofacial development is dependent on both endochondral and membranous ossification.

In skull vault, activated FGFR3 induces craniosiosynostosis. This disease consists of premature fusion of one or more of the cranial sutures. Two FGFR3 mutations cause specific craniosynostoses, Muenke syndrome and Crouzon syndrome with acanthosis nigricans. These diseases are an autosomal dominant hereditary disorder.

In long bone, FGFR3, when activated, exerts a negative regulatory influence mainly in the growth phase, in which it reduces the turnover necessary for bone elongation, the rate of cartilage template formation and disrupts chondrocyte proliferation and differentiation.

Abnormal FGFR3 overactivation or constitutive activation of FGFR3 leads to a severe disorganization of the growth plate cartilage. Gain of function mutants of FGFR3 (also called “constitutively active mutants of FGFR3”) disrupt endochondral ossification in a spectrum of skeletal dysplasias which include achondroplasia (ACH), the most common form of human dwarfism, hypochondroplasia (HCH), and thanatophoric dysplasia (TD), the most common form of lethal skeletal dysplasia. On the contrary, it has been shown that FGFR3 knock-out mice and humans without functional FGFR3 demonstrate skeletal overgrowth.

Therefore, FGFR3-related skeletal diseases (e.g. FGFR3-related skeletal dysplasias and FGFR3-related craniosiosynostosis) are the result of increased signal transduction from the activated receptor.

Among skeletal dysplasias, achondroplasia is of particular interest since it is one of the most common congenital diseases responsible for dwarfism, disorder characterized by short limbs relative to trunk. It is diagnosed by growth failure in the major axes of the long bones of extremities and typical physical features such as a large frontally projecting cranium and a short nose. This disease is an autosomal dominant hereditary disorder, but most of cases are found to be sporadic. Hypochondroplasia is also characterized by short stature with disproportionately short arms and legs. The skeletal features are very similar to achondroplasia but usually tend to be milder.

Current therapies of achondroplasia and hypochondroplasia include orthopedic surgeries such as leg lengthening and growth hormone therapy. However, leg lengthening inflicts a great pain on patients, and growth hormone therapy increases body height by means of periodic growth hormone injections starting from childhood. Further, growth ceases when injections are stopped.

Consequently, it is desirable to develop a new achondroplasia and hypochondroplasia therapy and to identify molecules suitable for treating achondroplasia and hypochondroplasia, as well as other FGFR3-related skeletal diseases such as FGFR3-related craniosiosynostosis.

Antagonists for FGFR3 receptor are well-known to those skilled in the art and include, e.g., anti-FGFR3 antibodies, for instance the antibodies described by Rauchenberger, R. et al. (J. Biol. Chem. 2003 Oct. 3; 278(40):38194-205.), Martinez-Torrecuadrada, J., et al. (Clin. Cancer Res. 2005 Sep. 1; 11(17):6280-90), Trudel S., et al., (Blood 2006 May 15; 107(10):4039-46.), Qing J. et al. (J. Clin. Invest. 2009, 119(5):1216-29), the anti-FGFR3 antibodies disclosed in IN2011CN02023, WO2010/111367, US 2010/0098696, WO2010/02862, WO2007/144893, WO2002/102973. Antagonists for FGFR3 receptor also include small chemical molecules, for instance those disclosed in WO2010/22169 (e.g. the compound of general formula 1 corresponding to 4,4′,4″,4′″-[carbonyl-bis[imino-5, 1,3-benzenetriyl bis-{carbonylimino}]3tetrakis-{benzene-1,3-disulfonic acid}), WO2007/26251, WO2005/47244, US2005/261307, as well as nucleic acid compounds for regulating/inhibiting FGFR3 expression described in WO2003/23004, US2007/049545 and WO2011/139843.

### SUMMARY OF THE INVENTION

The instant invention provides novel selective FGFR3 receptor antagonists with a high potency.

The compounds are of general formula (1):

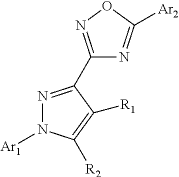

wherein the various substituents are as defined below.

The invention also pertains to compounds of general formula I as medicaments.

Compounds of general formula I are useful for the treatment of FGFR3-related skeletal diseases.

Compounds of general formula I are further useful for the treatment of cancer.

The invention further pertains to pharmaceutical compositions comprising a novel compound according to the invention.

The invention further pertains to a method for the treatment of a FGFR3-related skeletal disease, comprising administering to a subject in need thereof a compound as defined herein.

The invention further pertains to a method for the treatment of cancer comprising administering to a subject in need thereof a compound as defined herein.

### DETAILED DESCRIPTION OF THE INVENTION

### Definitions

Throughout the specification, several terms are employed and are defined in the following paragraphs.

As used herein, the terms “FGFR3”, “FGFR3 tyrosine kinase receptor” and “FGFR3 receptor” are used interchangeably throughout the specification and refer to all of the naturally-occurring isoforms of FGFR3.

As used herein, the expressions “constitutively active FGFR3 receptor variant”, “constitutively active mutant of the FGFR3” or “mutant FGFR3 displaying a constitutive activity” are used interchangeably and refer to a mutant of said receptor exhibiting a biological activity (i.e. triggering downstream signaling) in the absence of FGF ligand stimulation, and/or exhibiting a biological activity which is higher than the biological activity of the corresponding wild-type receptor in the presence of FGF ligand. Typically, the constitutively active FGFR3 receptor variant comprises at least one mutation selected from the group consisting of N540K, K650N, K650Q, S84L, R200C, N262H, G268C, Y278C, S279C and V381E

In the context of the present invention, the term “FGFR3-related skeletal disease” is intended to mean a skeletal disease that is caused by an abnormal increased activation of FGFR3, in particular by expression of a constitutively active mutant of the FGFR3 receptor, in particular a constitutively active mutant of the FGFR3 receptor as described above.

The FGFR3-related skeletal diseases are typically FGFR3-related skeletal dysplasias and FGFR3-related craniosynostosis.

The FGFR3-related skeletal dysplasias according to the invention may correspond to an inherited or to a sporadic disease.

As used herein, the term “FGFR3-related skeletal dysplasias” includes but is not limited to thanatophoric dysplasia type I, thanatophoric dysplasia type II, hypochondroplasia, achondroplasia and SADDAN (severe achondroplasia with developmental delay and acanthosis nigricans).

In a particular embodiment, the FGFR3-related skeletal dysplasia is caused by expression in the subject of a constitutively active FGFR3 receptor variant such as defined above.

In a particular embodiment, the FGFR3-related skeletal dysplasia is achondroplasia caused by expression of the G380R constitutively active mutant of the FGFR3 receptor.

In a particular embodiment, the FGFR3-related skeletal dysplasia is a hypochondroplasia caused by expression of the N540K, K650N, K650Q, S84L, R200C, N262H, G268C, Y278C, S279C, V381E, constitutively active mutant of the FGFR3 receptor.

In a particular embodiment, the FGFR3-related skeletal dysplasia is a thanatophoric dysplasia type I caused by expression of a constitutively active mutant of the FGFR3 receptor chosen from the group consisting of R248C, S248C, G370C, S371C; Y373C, X807R, X807C, X807G, X807S, X807W and K650M FGFR3 receptors.

In a particular embodiment, the FGFR3-related skeletal dysplasia is a thanatophoric dysplasia type II caused by expression of the K650E constitutively active mutant of the FGFR3 receptor.

In a particular embodiment, the FGFR3-related skeletal dysplasia is a severe achondroplasia with developmental delay and acanthosis nigricans caused by expression of the K650M constitutively active mutant of the FGFR3 receptor.

The FGFR3-related craniosynostosis according to the invention may correspond to an inherited or to a sporadic disease.

In a particular embodiment, the FGFR3-related craniosynostosis is Muenke syndrome caused by expression of the P250R constitutively active mutant of the FGFR3 receptor or Crouzon syndrome with acanthosis nigricans caused by expression of the A391G constitutively active mutant of the FGFR3 receptor.

As used herein the term “FGFR3 antagonist” refers to an agent (i.e. a molecule) which inhibits or blocks the activity of FGFR3. For instance, an antagonist of FGFR3 refers to a molecule which inhibits or blocks the activity of the FGFR3 receptor. Typically, the FGFR3 antagonists according to the invention act through direct interaction with the FGFR3 receptor.

The antagonists of the present invention act by blocking or reducing FGFR3 receptor functional activation. This may for example be achieved by interfering with FGF ligand binding to FGFR3 receptor or with ATP binding to “ATP binding site” of the FGFR3 receptor for preventing phosphorylation of tyrosine residues located towards the cytoplasmic domain (activation loop), i.e. on Tyr⁶⁴⁸ and Tyr⁶⁴⁷.

The antagonists according to the invention are capable of inhibiting or eliminating the functional activation of the FGFR3 receptor in vivo and/or in vitro. The antagonist may inhibit the functional activation of the FGFR3 receptor by at least about 10%, preferably by at least about 30%, preferably by at least about 50%, preferably by at least about 70, 75 or 80%, still preferably by 85, 90, 95, or 100%.

Typically, the antagonists according to the invention are more specific for FGFR3 versus FGFR1, 2 and 4. For instance the inhibitor constant “KI” of the antagonists for FGFR3 is at least 2, preferably 5, more preferably 10, times lower than the KI for at least one of FGFR1, 2 and 4.

Functional activation of the FGFR3 receptor may be readily assessed by the one skilled in the art according to known methods. Indeed, since the activated FGFR3 receptor is phosphorylated on tyrosine residues located towards the cytoplasmic domain, i.e. on Tyr⁶⁴⁸ and Tyr⁶⁴⁷, functional activation of the FGFR3 receptor may for example be assessed by measuring its phosphorylation.

For instance, analysis of ligand-induced phosphorylation of the FGFR3 receptor may be performed as described in Le Corre et al. (Org. Biomol. Chem., 8: 2164-2173, 2010).

Alternatively, receptor phosphorylation in cells may be readily detected by immunocytochemistry, immunohistochemistry and/or flow cytometry using antibodies which specifically recognize the modification. For instance phosphorylation of FGFR3 on the Tyr⁶⁴⁸ and Tyr⁶⁴⁷ residues may be detected by immunocytochemistry, immunohistochemistry and/or flow cytometry using monoclonal or polyclonal antibodies directed against phosphorylated Tyr⁶⁴⁸ and Tyr⁶⁴⁷-FGFR3.

Functional activation of the FGFR3 receptor may also be tested by using FGFR3-dependent cell lines (for instance BaF3 cell line). The FGFR3 antagonist activity of a compound is determined by measuring its ability to inhibit the proliferation of a FGFR3-dependent cell line (see methods described by Vito Guagnano et al., Journal of Medicinal Chemistry, 54: 7066-7083, 2011).

Further, FGFR3, when associated with its ligand, mediates signaling by activating the ERK and p38 MAP kinase pathways, and the STAT pathway. Therefore activation of the FGFR3 receptor may also be assessed by determining the activation of these specific pathways as described by Horton et al. (lancet, 370: 162-172, 2007).

As used herein, the term “subject” denotes a human or non-human mammal, such as a rodent, a feline, a canine, or a primate. Typically, the subject is a human being, more typically a child (i.e. a child who is growing up). Typically, when the subject to be treated is a child, the antagonist is administered during all or part of child growth period.

In the context of the invention, the term “treating” is used herein to characterize a therapeutic method or process that is aimed at (1) slowing down or stopping the progression, aggravation, or deterioration of the symptoms of the disease state or condition to which such term applies; (2) alleviating or bringing about ameliorations of the symptoms of the disease state or condition to which such term applies; and/or (3) reversing or curing the disease state or condition to which such term applies.

As used herein, the term “preventing” intends characterizing a prophylactic method or process that is aimed at delaying or preventing the onset of a disorder or condition to which such term applies.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Compounds of the Invention:

The invention relates to a compound of general formula (1)

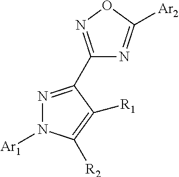

wherein
Ar₁ is phenyl unsubstituted or substituted with one to five R3 groups identical or different selected from, NR₅R₆, OR₅, COR₅, COOR₅, CONR₅R₆, NR₅COR₆,
—C≡CR₅ and 5-(1,2,3-triazolyl)-R₅;
Ar₂ is phenyl unsubstituted or substituted with one to five R₄ groups identical or different selected from Halo, NO₂, NR₅R₆, OR₅, COR₅, COOR₅, CONR₅R₆ and NR₅COR₆;
R₁ is NR₅R₆ or NR₅COR₆;
R₂ is COOR₅, CONR₅R₆ or CONR₅OR₆;
R₅ and R₆ identical or different are selected from H, alkyl, cycloalkyl, fluoroalkyl, phenyl, benzyl, pyridyl, CO-alkyl, CO-fluoroalkyl and CO-phenyl, CO-benzyl NH-alkyl, NH-fluoroalkyl, NH-phenyl and NH-benzyl;
wherein the phenyl, benzyl and pyridyl groups are unsubstituted or substituted by one or more R₇ groups selected from alkyl, O-alkyl, cycloalkyl, fluoroalkyl and phenyl or a pharmaceutically acceptable salt thereof with the exclusion of the following compounds:
   R1 is NH COCH3, R2 is CO2CH3, Ar1 is 3-methoxyphenyl is and Ar2 is phenyl
   R1 is NH COCH3, R2 is CO2CH3, Ar1 is 3-methoxyphenyl is and Ar2 is 4-methoxyphenyl
   R1 is NH COCH3, R2 is CO2CH3, Ar1 is 3-methoxyphenyl is and Ar2 is 4-nitrophenyl.

In the above general formula (1):
Alkyl denotes a straight-chain or branched group containing 1, 2, 3, 4, 5 or 6 carbon atoms. Examples of suitable alkyl radicals are methyl, ethyl, n-propyl, isopropyl, n-butyl, isobutyl, sec-butyl, tert-butyl, etc.
Alkyl preferably comprises not more than 4 carbon atoms.
Fluoroalkyl denotes a straight-chain or branched group containing 1, 2, 3, 4, 5 or 6 carbon atoms, wherein one or more carbon atoms are replaced with a fluorine atom. Treferably all tha carbon atoms are replaced s with fluorine atoms in which case the fluoroalkyl group is perfluoroalkyl.
Cycloaklyl denotes a cyclic alkyl group comprising from 3 to 12 carbon atoms that may be mono- or bicyclic or bridged.
Preferred groups are cyclopropyl, cyclopentyl, cyclohexyl and adamantyl.
Halo denotes a halogen atom selected from the group consisting of fluoro, chloro, bromo and iodo, in particular bromo.

Preferred compounds of general formula I are those wherein R₁ is selected from NH₂, NH-alkyl, N-(alkyl)₂, NHCO-alkyl, N(CO-alkyl)₂, NH-fluoroalkyl, N-(fluoroalkyl)₂, NHCO-fluoroalkyl, N—(CO-trifluoroalkyl)₂ and R₂ is selected from COOH, COO-alkyl, CO—NH-alkyl and CO—NH—O-alkyl.

In a preferred embodiment, Ar₁ is unsubstituted or substituted by one R₃ group selected from OR₅, COOH, COOR₅, CONHR₆, —C≡CH, and a group of formula:

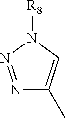

R₅ and R₆ identical or different are selected from (cyclo) alkyl, fluoroalkyl, phenyl, benzyl, pyridyl, CO-alkyl, CO-fluoroalkyl and CO-phenyl, CO-benzyl NH-alkyl, NH-fluoroalkyl, NH-phenyl and NH-benzyl, R₈ is selected from phenyl, benzyl, pyridyl, wherein phenyl, benzyl and pyridyl are unsubstituted or substituted by one or more groups selected from alkyl, trifluoro-alkyl and O-alkyl.

A preferred subgroup of compounds according to the invention consists of compounds of general formula II:

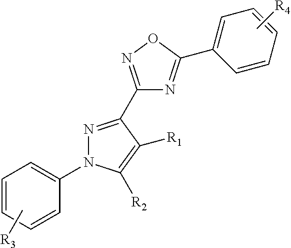

wherein
R₁ is selected from NH₂, NH Alkyl, N((cyclo)alkyl)₂, NHCO-(cyclo)alkyl, N (CO-(cyclo) alkyl)₂ and NH-fluoroalkyl;
R₂ is selected from COOR₅, CONR₅R₆ and CO—NH-Oalkyl
R₃ is selected from H, OR₅, COOH, COOR₅, CONHR₆ and —C≡CR₅, and a group of formula:

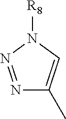

R₅ and R₆ identical or different are selected from H, (cyclo) alkyl, fluoroalkyl, phenyl, benzyl, pyridyl, CO-alkyl, CO-fluoroalkyl and CO-phenyl, CO-benzyl, NH-alkyl, NH-fluoroalkyl, NH-phenyl and NH-benzyl,
R₈ is selected from phenyl, benzyl, pyridyl, wherein phenyl, benzyl and pyridyl are unsubstituted or substituted by one or more groups selected from alkyl, trifluoro-alkyl and O-alkyl;
R₄ is selected from the group consisting of H, Halo, NO₂, NH₂, NH-(cyclo) alkyl and N(-(cyclo) alkyl)₂,
R₅ and R₆ identical or different are selected from H, alkyl, cycloalkyl, fluoroalkyl, phenyl, CO-(cyclo) alkyl, CO-fluoroalkyl and CO-phenyl,
or a pharmaceutically acceptable salt thereof, with the exclusion of the following compounds:
   R1 is NH COCH3, R2 is CO2CH3, Ar1 is 3-methoxyphenyl is and Ar2 is phenyl
   R1 is NH COCH3, R2 is CO2CH3, Ar1 is 3-methoxyphenyl is and Ar2 is 4-methoxyphenyl
   R1 is NH COCH3, R2 is CO2CH3, Ar1 is 3-methoxyphenyl is and Ar2 is 4-nitrophenyl.

In a preferred embodiment R₃ is in the meta position and R₄ is in the ortho or para position.

Compounds of formula (I) may be prepared using conventional procedures such as by the following illustrative methods in which the various substituents are as previously defined for the compounds of the formula (I) unless otherwise stated.

The compounds provided herein may be prepared from known or commercially available starting materials and reagents by one skilled in the art of organic synthesis. Such procedures include recrystallization, column chromatography or HPLC.

The following schemes are presented with details as to the preparation of representative compounds of the invention.

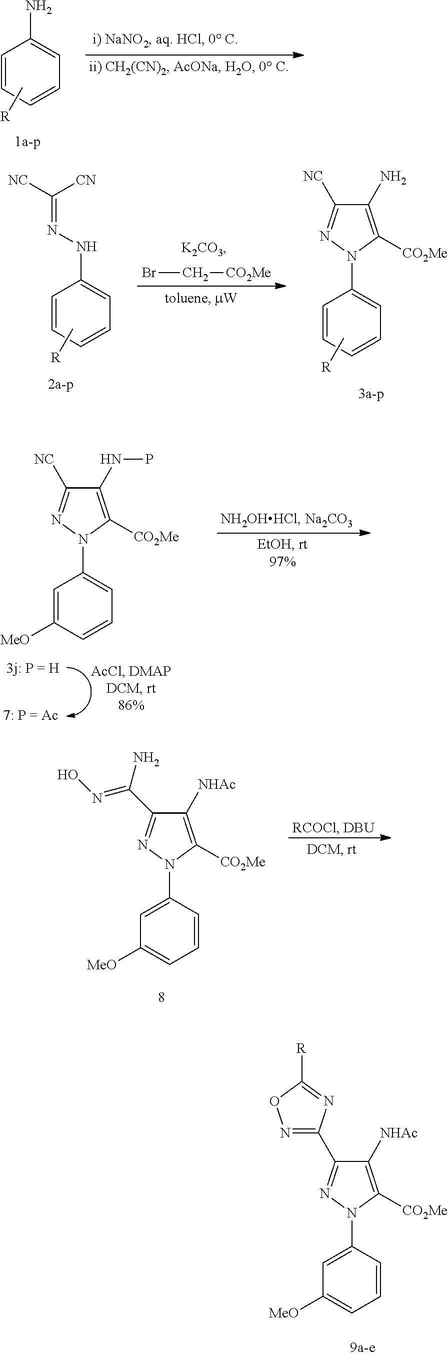

| Entry | R | Product ^{a} | Yield^{b} (%) |
|---|---|---|---|
| 1 | Me | 9a | 71 |
| 2 | 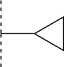 | 9b | 77^{c} |
| 3 | Ph | 9c | 64 |
| 4 | 4-MeOPh | 9d | 81 |
| 5 | 4-NO₂Ph | 9e | 83^{c} |

^{a} Reaction conditions: N-hydroxyamidine 8 (1 equiv.), RCOCl (1.1 equiv.), DBU (2 equiv.), DCM, rt, 16 h.
^{b}Yield of isolated product.
^{c}Completion of the reaction was reached after 4 h.

General Procedure I for the Synthesis of the Aryl-Hydrazones 2a-p

To an ice-cooled solution of the aniline 1 (1 equiv.) in water (5 mL/mmol) were successively added dropwise 37% aq. HCl (11 equiv.) and 1 M aq. NaNO₂ (1 equiv.). The mixture was stirred 30 min and then dropwise added to a solution of malononitrile (1.5 equiv.) and sodium acetate (31 equiv.) in water (8.5 mL/mmol of aniline) with continous stirring and cooling to 0° C. After 2 h, the insoluble hydrazone was filtered off and washed with water. The precipitate was dissolved with EtOAc and washed with brine. The organic layer was dried (MgSO₄) and concentrated in vacuo to afford the desired hydrazone which was used without purification (unless indicated).

General Procedure II for the Synthesis of the Pyrazoles 3a-p

A mixture of hydrazone 2 (1 equiv.), potassium carbonate (7.5 equiv.), methyl bromoacetate (2.7 equiv.) in anhydrous solvent (3 mL/mmol) was irradiated at 120° C. (power imput: 90 W) for 8 to 45 min. The reaction mixture was cooled to rt and concentrated in vacuo. The resulting residue was dissolved in DCM and washed with brine. The organic layer was dried (MgSO₄) then concentrated in vacuo. Flash chromatography afforded the desired pyrazole.

### Methyl 4-acetamido-3-cyano-1-(3-methoxyphenyl)-1H-pyrazole-5-carboxylate (7)

To an ice-cooled solution of the aminopyrazole 3j (2 g, 7.35 mmol) in DCM (34 mL) were successively added DMAP (942 mg, 7.72 mmol, 1.05 equiv.) and acetyl chloride (530 μL, 7.42 mmol, 1 equiv.). The reaction mixture was stirred at rt for 18 h. After dilution with DCM (150 mL), the organic layer was successively washed with 0.5 N HCl (30 mL), satd. aq. NaHCO₃ (50 mL) and brine (50 mL). The organic layer was dried (MgSO₄) and concentrated in vacuo. Flash chromatography (DCM/MeOH 98:2) afforded 7 as a grey solid (1.97 g, 86%):

### Methyl 4-acetamido-3-(N′-hydroxycarbamimidoyl)-1-(3-methoxyphenyl)-1H-pyrazole-5-carboxylate (8)

A mixture of the aminopyrazole 7 (1.97 g, 6.27 mmol), hydroxylamine hydrochloride (2.19 g, 31.5 mmol, 5 equiv.) and Na₂CO₃ (1.68 g, 15.8 mmol, 2.5 equiv.) in EtOH (125 mL) was heated at 80° C. for 1 h. After cooling to rt, the solution was concentrated in vacuo. The resulting residue was dissolved in DCM (250 mL) and washed with brine (50 mL). The organic layer was dried (MgSO₄) and concentrated in vacuo to afford the N-hydroxyamidine 8 as a yellow solid (2.10 g, 97%) which was used without further purification:

General Procedure III for the Synthesis of the Pyrazolo-Oxadiazoles 9a-e

To an ice-cooled solution of the N-hydroxyamidine 8 (1 equiv.) in DCM (11.5 mL/mmol) were added DBU (2 equiv.) and acyl chloride (1.1 equiv.). The reaction mixture was stirred at rt for 4-16 h, diluted with DCM (140 mL/mmol), and the pH was adjusted to 2 with 1 M aq. HCl. The organic layer was washed with satd. aq. NaHCO₃ until pH 8, dried (MgSO₄) and concentrated in vacuo. Flash chromatography afforded the desired pyrazolo-oxadiazole.

Pharmaceutically acceptable salts of the compounds of formula (I) include the acid addition and base salts thereof. Suitable acid addition salts are formed from acids, which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate and xinafoate salts.

For a review on suitable salts, see “Handbook of Pharmaceutical Salts: Properties, Selection, and Use” by Stahl and Wermuth (Wiley-VCH, Weinheim, Germany, 2002). Pharmaceutically acceptable salts of compounds of formula (I) may be prepared by one or more of three methods:
(i) by reacting the compound of formula (1) with the desired acid or base;
(ii) by removing an acid- or base-labile protecting group from a suitable precursor of the compound of formula (1) or by ring-opening a suitable cyclic precursor, for example, a lactone or lactam, using the desired acid or base; or
(iii) by converting one salt of the compound of formula (1) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionization in the resulting salt may vary from completely ionized to almost non-ionized.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water. Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components, which may be in stoichiometric or non-stoichiometric amounts. The resulting complexes may be ionised, partially ionized, or non-ionized. For a review of such complexes, see J Pharm Sci, 64 (8), 1269-1288 by Haleblian (August 1975).

Hereinafter all references to compounds of formula (I) include references to salts, solvates and complexes thereof and to solvates and complexes of salts thereof.

The compounds of the invention include compounds of formula (I) as hereinbefore defined, including all polymorphs and crystal habits thereof, prodrugs and isomers thereof whenever relevant

So-called ‘pro-drugs’ of the compounds of formula (I) are also within the scope of the invention. Thus certain derivatives of compounds of formula (I) which may have little or no pharmacological activity themselves can, when administered into or onto the body, be converted into compounds of formula (I) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T. Higuchi and W. Stella) and ‘Bioreversible Carriers in Drug Design’, Pergamon Press, 1987 (ed. E. B Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in “Design of Prodrugs” by H. Bundgaard (Elsevier, 1985).

Some examples of prodrugs in accordance with the invention include amides thereof, for example, a compound wherein, as the case may be the hydrogen of the amino functionality of the compound of formula (1) is/are replaced by (C₁-C₁₀)alkanoyl.

Further examples of replacement groups in accordance with the foregoing examples and examples of other prodrug types may be found in the aforementioned references. Moreover, certain compounds of formula (I) may themselves act as prodrugs of other compounds of formula (I).

Also included within the scope of the invention are metabolites of compounds of formula (I), that is, compounds formed in vivo upon administration of the drug, such as a primary amino derivatives thereof or phenol derivative thereof, or carboxylic acid derivative

Pharmaceutically acceptable solvates in accordance with the invention include those wherein the solvent of crystallization may be isotopically substituted, e.g. D₂O.

### Therapeutic Applications:

The compounds of formula (I), their pharmaceutically acceptable salts and/or derived forms, are valuable pharmaceutically active compounds, which are suitable for the therapy and prophylaxis for use in the treatment or prevention of FGFR3-related diseases.

Accordingly, the invention further pertains to compounds of formula (I) or of formula (II) as defined above, for use as medicaments, namely for antagonizing the fibroblast growth factor receptor 3 (FGFR3), which are useful for the treatment or the prevention of FGFR3-related diseases, such as cancers or FGFR3-related skeletal diseases.

For example, the compounds of the invention are useful in the treatment of cancer. Example cancers include bladder cancer, breast cancer, cervical cancer, colorectal cancer, endometrial cancer, gastric cancer, head and neck cancer, kidney cancer, liver cancer, lung cancer (e.g., adenocarcinoma, small cell lung cancer and non-small cell lung carcinomas), ovarian cancer, prostate cancer, esophageal cancer, gall bladder cancer, ovarian cancer, pancreatic cancer (e.g. exocrine pancreatic carcinoma), stomach cancer, thyroid cancer, skin cancer (e.g., squamous cell carcinoma). Further example cancers include hematopoietic malignancies such as leukemia, multiple myeloma, chronic lymphocytic lymphoma, adult T cell leukemia, B-cell lymphoma, acute myelogenous leukemia, Hodgkin's or non-Hodgkin's lymphoma, myeloproliferative neoplasms (e.g., polycythemia vera, essential thrombocythemia, and primary myelofibrosis), Waldenstrom's Macroglubulinemia, hairy cell lymphoma, and Burkett's lymphoma. Other cancers treatable with the compounds of the invention include glioblastoma, melanoma, and rhabdosarcoma.

The compounds of the present invention are also suitable for treating FGFR3-related diseases. The FGFR3-related skeletal diseases are typically FGFR3-related skeletal dysplasias and FGFR3-related craniosynostosis. The FGFR3-related skeletal dysplasias according to the invention may correspond to an inherited or to a sporadic disease and include hanatophoric dysplasia type I, thanatophoric dysplasia type II, hypochondroplasia, achondroplasia and SADDAN (severe achondroplasia with developmental delay and acanthosis nigricans).

Typically, a compound of the invention is administered in a therapeutically effective amount. By “therapeutically effective amount” is meant a sufficient amount of the antagonist of the invention to treat and/or to prevent the disease at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disease being treated and the severity of the disease; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day. These dosages are based on an average human subject having a weight of about 65 kg to 70 kg. The physician will readily be able to determine doses for subjects whose weight falls outside this range, such as infants and the elderly.

A further aspect of the present invention is a pharmaceutical composition comprising a compound of the invention and a pharmaceutically acceptable carrier or excipient.

“Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The compounds of the invention may also be combined with sustained-release matrices, such as biodegradable polymers, to form therapeutic compositions.

In addition to the compounds of the invention, the pharmaceutical composition may further comprise and additional active ingredient for the treatment of FGFR3-related skeletal diseases.

In some embodiments, the pharmaceutical composition of the invention typically comprises a combination of a compound of the invention and an additional active ingredient for the treatment of FGFR3-related skeletal diseases and a pharmaceutically acceptable carrier.

In addition to the compounds of the invention, the pharmaceutical composition may further comprise and additional active ingredient for the treatment of cancer.

In some embodiments, the pharmaceutical composition of the invention typically comprises a combination of a compound of the invention and an additional active ingredient for the treatment of cancer and a pharmaceutically acceptable carrier.

Thus, a compound of the invention may be formulated as a pharmaceutical composition for oral, buccal, intranasal, parenteral (e. g. intravenous, intramuscular or subcutaneous), topical, or rectal administration or in a form suitable for administration by inhalation or insufflation.

For oral administration, the pharmaceutical composition may take the form of, for example, a tablet or capsule prepared by conventional means with a pharmaceutically acceptable excipient such as a binding agent (e. g., pregelatinized maize starch, polyvinylpyrrolidone or hydroxypropyl methylcellulose); filler (e. g., lactose, microcrystalline cellulose or calcium phosphate); lubricant (e. g., magnesium stearate, talc or silica); disintegrant (e. g., potato starch or sodium starch glycolate); or wetting agent (e. g., sodium lauryl sulphate). The tablets may be coated by methods well known in the art. Liquid preparations for oral administration may take the form of a, for example, solution, syrup or suspension, or they may be presented as a dry product for constitution with water or other suitable vehicle before use.

Such liquid preparations may be prepared by conventional means with a pharmaceutically acceptable additive such as a suspending agent (e. g., sorbitol syrup, methyl cellulose or hydrogenated edible fats); emulsifying agent (e. g., lecithin or acacia); non-aqueous vehicle (e. g., almond oil, oily esters or ethyl alcohol); and preservative (e. g., methyl or propyl p-hydroxybenzoates or sorbic acid).

For buccal administration, the composition may take the form of tablets or lozenges formulated in conventional manner. A compound of the present invention may also be formulated for sustained delivery according to methods well known to those of ordinary skill in the art.

Examples of such formulations can be found in U.S. Pat. Nos. 3,538,214, 4,060,598, 4,173,626, 3,119,742, and 3,492,397, which are herein incorporated by reference in their entirety.

A compound of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain a formulating agent such as a suspending, stabilizing and/or dispersing agent. Alternatively, the active ingredient may be in powder form for reconstitution with a suitable vehicle, e. g., sterile pyrogen-free water, before use parenteral formulations are typically aqueous solutions which may contain excipients such as salts, carbohydrates and buffering agents (typically to a pH of from 3 to 9), but, for some applications, they may be more suitably formulated as a sterile non-aqueous solution or as a dried form to be used in conjunction with a suitable vehicle such as sterile, pyrogen-free water.

The following examples illustrate the preparation of the compounds of the formula (1) and their pharmacological properties

### EXAMPLES

### 1. Methyl 4-acetamido-3-(5-(4-bromophenyl)-1,2,4-oxadiazol-3-yl)-1-(3-methoxyphenyl)-1H-pyrazole-5-carboxylate

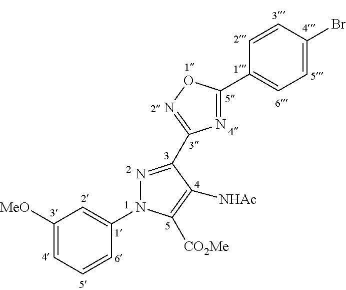 [SMILES: COC(=O)c1c(NC(C)=O)c(-c2noc(-c3ccc(Br)cc3)n2)nn1-c1cccc(OC)c1]

Mp: 224° C.; Rf 0.36 (CHCl₃/MeOH 98:2); 1H NMR (500 MHz, CDCl₃): δ 8.35 (br s, 1H, NHAc), 8.09 (d, J=8.5 Hz, 2H, H-2′″, H-6′″), 7.69 (d, J=8.5 Hz, 2H, H-3′″, H-5′″), 7.33 (dd, J=8.0 Hz, J=8.0 Hz, 1H, H-5′), 7.08-7.02 (m, 2H, H-2′, H-6′), 6.99-6.95 (m, 1H, H-4′), 3.83 (s, 3H, OCH₃), 3.78 (s, 3H, CO₂CH₃), 2.25 (s, 3H, COCH₃); 13C NMR (125 MHz, CDCl₃): δ 175.2 (C-5″), 167.9 (COCH₃), 163.8 (C-3″), 160.5 (CO₂CH₃), 160.1 (C-3′), 140.8 (C-1′), 132.8 (2C, C-3′″, C-5′″), 132.4 (Cpyr), 129.9 (2C, C-2′″, C-6′″), 129.7 (C-5′), 128.5 (C-1′″), 128.4 (Cpyr), 122.7 (C-4′″), 122.4 (Cpyr), 117.8 (C-6′), 115.7 (C-4′), 111.3 (C-2′), 55.8 (OCH₃), 52.7 (CO₂CH₃), 23.8 (COCH₃); IR (υ, cm-1): 3240 (NH), 1731 (C═O), 1678 (NHC═O), 1606, 1583, 1563, 1551 (C═C, C═N), 1470 (CH), 1252 (C—O), 1242 (C—O), 1088, 1011; MS (ESI): m/z=512, 514 [M+H]+; HRMS (TOF MS ES): calc. for C₂₂H₉N₅O₅⁷⁹Br [M+H]+ 512.0570, found 512.0576.

### 2. Methyl 4-acetamido-1-(3-methoxyphenyl)-3-(5-(4-nitrophenyl)-1,2,4-oxadiazol-3-yl)-1H-pyrazole-5-carboxylate

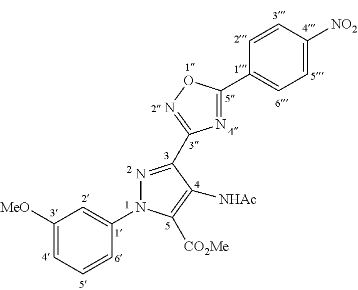 [SMILES: COC(=O)c1c(NC(C)=O)c(-c2noc(-c3ccc([N+](=O)[O-])cc3)n2)nn1-c1cccc(OC)c1]

Mp: 248° C.; Rf 0.13 (CHCl₃/MeOH 98:2); Rf 0.48 (cyclohexane/acetone 1:1); 1H NMR (500 MHz, CDCl₃): δ 8.46-8.37 (m, 4H, H-2′″, H-3′″, H-5′″, H-6′″), 8.25 (br s, 1H, NHAc), 7.34 (dd, J=8.0 Hz, J=8.0 Hz, 1H, H-5′), 7.09-7.02 (m, 2H, H-2′, H-6′), 7.02-6.97 (m, 1H, H-4′), 3.84 (s, 3H, OCH₃), 3.78 (s, 3H, CO₂CH₃), 2.26 (s, 3H, COCH₃); 13C NMR (125 MHz, CDCl₃): δ 173.9 (C-5″), 167.9 (COCH₃), 164.3 (C-3″), 160.2 and 160.1 (C-3′, CO₂CH₃), 150.7 (C-4′″), 140.8 (C-1′), 132.5 (Cpyr), 129.7 (3C, C-5′, C-2′″, C-6′″), 129.1 (C-1′″), 128.3 (Cpyr), 124.6 (2C, C-3′″, C-5′″), 122.9 (Cpyr), 117.9 (C-6′), 115.7 (C-4′), 111.5 (C-2′), 55.8 (OCH₃), 52.8 (CO₂CH₃), 23.8 (COCH₃); IR (υ, cm-1): 3245 (NH), 1727 (C═O), 1676 (NHC═O), 1607, 1574, 1560 (C═C, C═N), 1529 (NO₂), 1495 (CH), 1347 (NO₂), 1242 (C—O), 1132, 1048, 1032; MS (ESI): m/z=479 [M+H]+; HRMS (ESI): calc. for C₂₂H₁₉N₆O₇ [M+H]+ 479.1315, found 479.1331.

### 3. Methyl 4-acetamido-3-(5-(4-(dimethylamino)phenyl)-1,2,4-oxadiazol-3-yl)-1-(3-methoxyphenyl)-1H-pyrazole-5-carboxylate

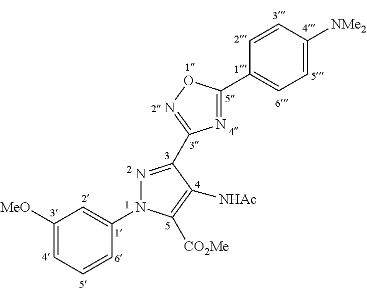 [SMILES: COC(=O)c1c(NC(C)=O)c(-c2noc(-c3ccc(N(C)C)cc3)n2)nn1-c1cccc(OC)c1]

Mp: 196° C.; Rf 0.36 (EtOAc/cyclohexane 9:1); 1H NMR (500 MHz, CDCl₃): δ 8.65 (br s, 1H, NHAc), 8.04 (d, J=8.5 Hz, 2H, H-2′″, H-6′″), 7.36-7.27 (dd, J=7.5 Hz, J=7.5 Hz, 1H, H-5′), 7.13-7.01 (m, 2H, H-2′, H-6′), 6.99-6.92 (m, 1H, H-4′), 6.71 (d, J=8.5 Hz, 2H, H-3′″, H-5′″), 3.82 (s, 3H, OCH₃), 3.78 (s, 3H, CO₂CH₃), 2.94 (s, 6H, NMe₂), 2.24 (s, 3H, COCH₃); 13C NMR (125 MHz, CDCl₃): δ 176.5 (C-5″), 167.8 (COCH₃), 163.3 (C-3″), 160.9 (CO₂CH₃), 160.1 (C-3′), 153.5 (C-4′″), 140.9 (C-1′), 132.3 (Cpyr), 130.2 (2C, C-2′″, C-6′″), 129.6 (C-5′), 128.4 (Cpyr), 121.9 (Cpyr), 117.7 (C-6′), 115.6 (C-4′), 111.6 (2C, C-3′″, C-5′″), 111.1 (C-2′), 110.5 (C-1′″), 55.8 (OCH₃), 52.7 (CO₂CH₃), 40.2 (NMe₂), 23.8 (COCH₃); IR (v, cm-1): 3297 (NH), 3066, 2972, 2833 (CH), 1727 (C═O), 1675 (NHC═O), 1614, 1586, 1565 (C═C, C═N), 1515, 1455 (CH), 1372, 1284, 1234 (C—O), 1188, 1048, 1025; MS (ESI): m/z=477 [M+H]+; HRMS (ESI): calc. for C₂₄H₂₅N₆O₅ [M+H]+ 477.1886, found 477.1901.

### 4. Methyl 4-acetamido-1-(3-methoxyphenyl)-3-(5-phenyl-1,2,4-oxadiazol-3-yl)-1H-pyrazole-5-carboxylate

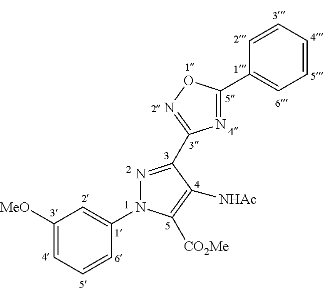 [SMILES: COC(=O)c1c(NC(C)=O)c(-c2noc(-c3ccccc3)n2)nn1-c1cccc(OC)c1]

Mp 186-188° C. (EtOH); Rf0.31 (DCM/MeOH 98:2); 1H NMR (500 MHz, DMSO-d₆) δ 9.81 (br s, 1H, NHAc), 8.24-8.15 (m, 2H, H-2′″, H-6′″), 7.78-7.72 (m, 1H, H-4′″), 7.71-7.64 (m, 2H, H-3′″, H-5′″), 7.50-7.42 (m, 1H, H-5′), 7.16-7.05 (m, 3H, H-2′, H-4′, H-6′), 3.83 (s, 3H, OCH₃), 3.73 (s, 3H, CO₂CH₃), 2.08 (s, 3H, COCH₃); 13C NMR (125 MHz, DMSO-d₆) δ 175.0 (C-5″), 168.5 (COCH₃), 162.7 (C-3″), 159.4 (C-3′), 158.7 (CO₂CH₃), 140.5 (C-1′), 135.0 (Cpyr), 133.5 (C-4′″), 129.8 (C-5′), 129.6 (2C, C-3′″, C-5′″), 129.2 (Cpyr), 127.9 (2C, C-2′″, C-6′″), 123.2 and 123.1 (C-1′″, Cpyr), 117.3 (C-6′), 114.9 (C-4′), 110.8 (C-2′), 55.5 (OCH₃), 52.3 (CO₂CH₃), 22.7 (COCH₃); IR υ 3251 (NH), 1726 (C═O), 1671 (HNC═O), 1607, 1584, 1550 (C═C, C═N), 1493, 1468, 1453, 1476, 1369, 1248, 1224, 1127, 1048, 1031; MS (ESI) m/z 434 [M+H]+; HRMS (ESI) m/z [M+H]+ Calcd for C₂₂H₂₀N₅O₅ 434.1459, Found 434.1455.

### 5: Pharmacological Activities of the Compounds of the Invention are Illustrated in the Table Hereunder

The pharmacological activity of the compounds was assessed by the following tests:
HEK 293 VNR cells were seeded in 6-wells plate 48 to 72 h before cell transfection
At confluence 80%, cell transfection was performed with a ratio of 2 μg of plasmid with FGFR3-K650E^{TD} for 4 μL of JetPEI per 6-well dish
6 to 8 h after cell transfection, cells were washed and exposed (in accordance with previous results) to 5 non-toxic dose range of compound during 16 h at 37° C. in 5% CO₂ atmosphere
Exposed cells were scrapped in 400 μL of RIPA buffer (Lysis buffer)
Cells extracts were centrifuged 11 000 rpm for 20 minutes
Rotation of supernatant was performed on wheel over night at 4° C. with 3 μL of FGFR3 antibody
30 μL beads were added to the mix <<FGFR3 antibody-supernantant>> for 4 to 5 hours
beads were washed 3× with 500 μL of lysis buffer (centrifugation 3000 rpm, 2 minutes)
Elution was performed with 30 μL of RIPA buffer containing SDS 10% and blue bromophenol 1% at 95° C., 10 min
western-blotting was performed
Quantification of immunoreactivities was carried out by FIDJI software (advanced ImageJ software)
IC50 were determined by GraphPad Prism® software

| Cpd | | M (Molar | |
|---|---|---|---|
| N° | Structure | mass) | % in vitro inhibition |
| 24 | 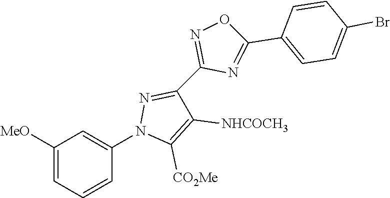 | 512.31 | 72, 14 IC50 = 3, 5 μM In vitro, WT IC50 = 150 nM in cellulo |
| 25 | 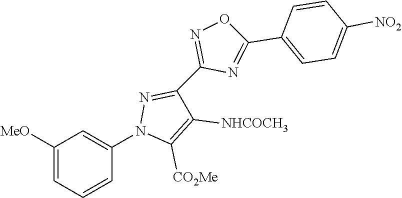 | 478.41 | 70, 30 IC50 = 7, 6 μM In vitro, WT inhibition = 0% @ 500 nM in cellulo, K650E |
| 27 | 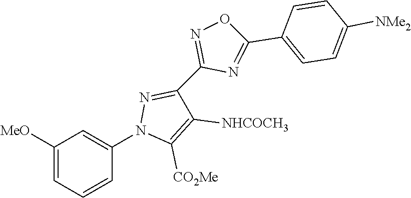 | 476.48 | 61, 98 |
| 29 | 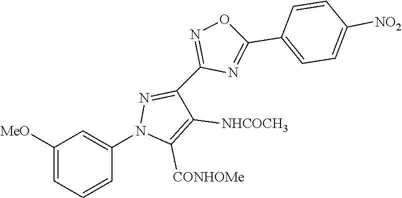 | 493.42 | 85, 00 |
| 30 | 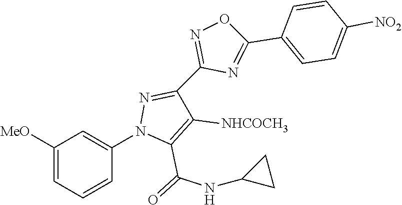 | 503.46 | 69, 53 |
| 31 | 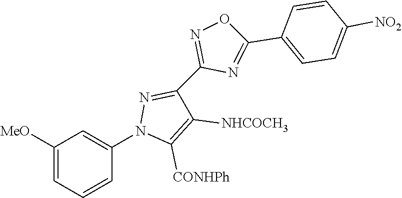 | 539.49 | 71, 87 |
| 32 | 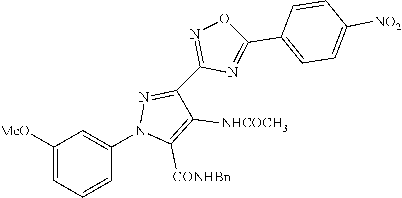 | 553.52 | 89, 14 IC50 = 0, 77 μM |
| 33 | 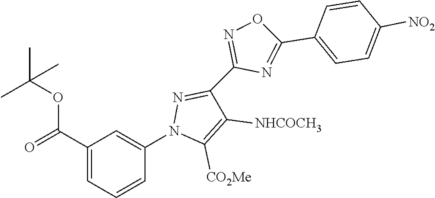 | 548.50 | 61, 77 |
| 37 | 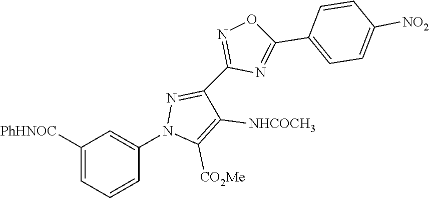 | 567.50 | 75, 62 IC50 = 1, 8 μM |
| 42 | 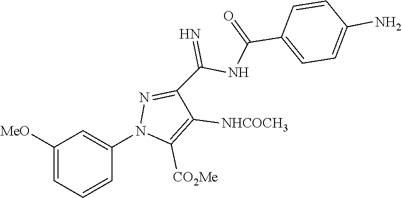 | 450.44 | 61, 24 |
| 62 | 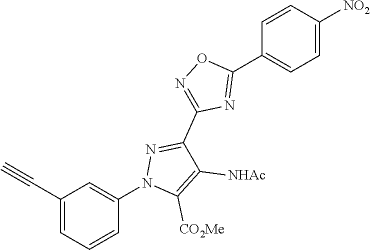 [SMILES: C#Cc1cccc(-n2nc(-c3noc(-c4ccc([N+](=O)[O-])cc4)n3)c(NC(C)=O)c2C(=O)OC)c1] | 472.40 | 74, 90 |
| 63 | 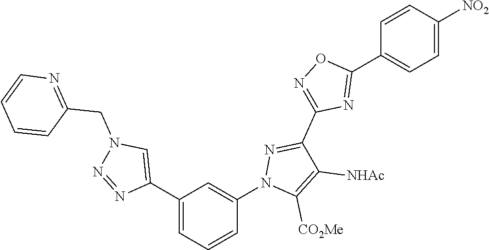 [SMILES: COC(=O)c1c(NC(C)=O)c(-c2noc(-c3ccc([N+](=O)[O-])cc3)n2)nn1-c1cccc(-c2cn(Cc3ccccn3)nn2)c1] | 606.54 | 71, 42 |
| 64 | 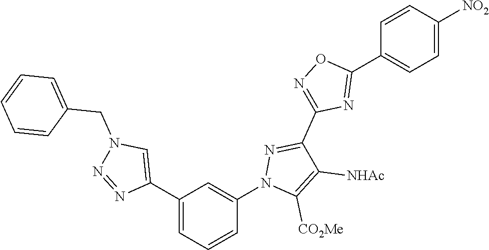 [SMILES: COC(=O)c1c(NC(C)=O)c(-c2noc(-c3ccc([N+](=O)[O-])cc3)n2)nn1-c1cccc(-c2cn(Cc3ccccc3)nn2)c1] | 605.56 | 92, 30 IC50 = 2 μM |
| 65 | 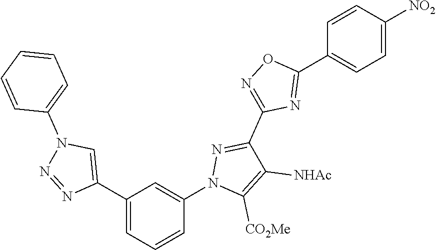 [SMILES: COC(=O)c1c(NC(C)=O)c(-c2noc(-c3ccc([N+](=O)[O-])cc3)n2)nn1-c1cccc(-c2cn(-c3ccccc3)nn2)c1] | 591.53 | 71, 08 |
| 66 | 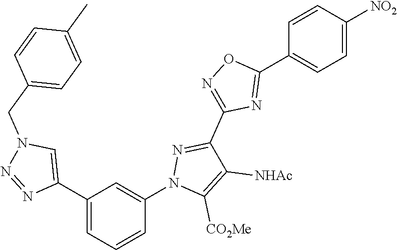 [SMILES: COC(=O)c1c(NC(C)=O)c(-c2noc(-c3ccc([N+](=O)[O-])cc3)n2)nn1-c1cccc(-c2cn(Cc3ccc(C)cc3)nn2)c1] | 619.58 | 81, 95 |
| 67 | 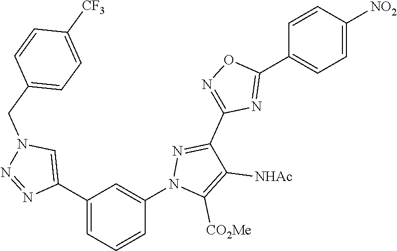 [SMILES: COC(=O)c1c(NC(C)=O)c(-c2noc(-c3ccc([N+](=O)[O-])cc3)n2)nn1-c1cccc(-c2cn(Cc3ccc(C(F)(F)F)cc3)nn2)c1] | 673.55 | 54, 28 |
| 68 | 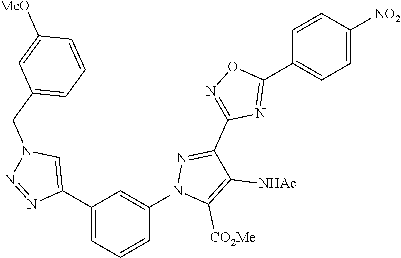 [SMILES: COC(=O)c1c(NC(C)=O)c(-c2noc(-c3ccc([N+](=O)[O-])cc3)n2)nn1-c1cccc(-c2cn(Cc3cccc(OC)c3)nn2)c1] | 635.58 | 52, 26 |
| 79 | 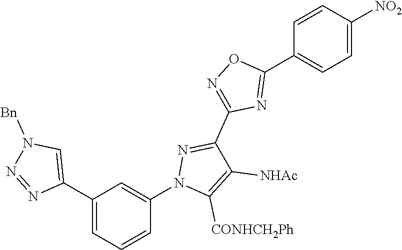 | 680.67 | 21 @ 10 μM 97 @ 50 μM |
| 103 | 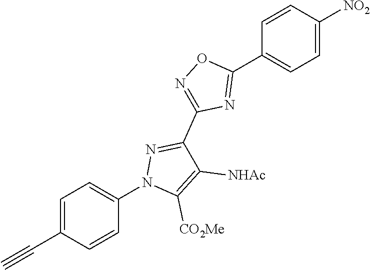 [SMILES: C#Cc1ccc(-n2nc(-c3noc(-c4ccc([N+](=O)[O-])cc4)n3)c(NC(C)=O)c2C(=O)OC)cc1] | 472.41 | 55, 64 |
| 116 | 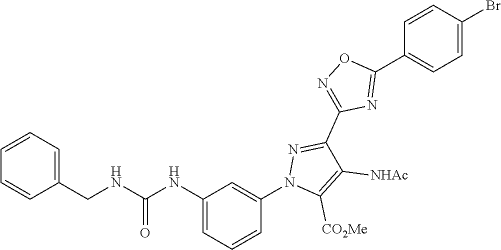 [SMILES: COC(=O)c1c(NC(C)=O)c(-c2noc(-c3ccc(Br)cc3)n2)nn1-c1cccc(NC(=O)NCc2ccccc2)c1] | 630.46 | 57, 96 |
| 134 | 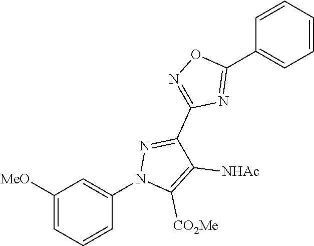 [SMILES: COC(=O)c1c(NC(C)=O)c(-c2noc(-c3ccccc3)n2)nn1-c1cccc(OC)c1] | 433.41 | 87, 41 |
| 135 | 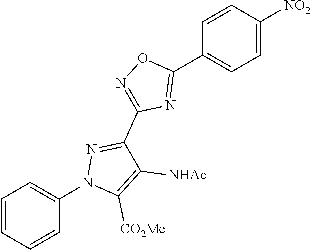 [SMILES: COC(=O)c1c(NC(C)=O)c(-c2noc(-c3ccc([N+](=O)[O-])cc3)n2)nn1-c1ccccc1] | 448.38 | 64, 50 IC₅₀: NC (in vitro WT) |
| 136 | 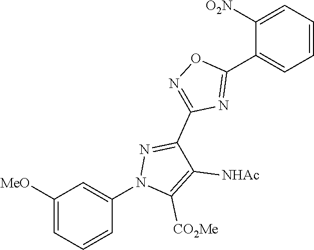 [SMILES: COC(=O)c1c(NC(C)=O)c(-c2noc(-c3ccccc3[N+](=O)[O-])n2)nn1-c1cccc(OC)c1] | 478.41 | 72, 68 IC₅₀: NC (in vitro WT) |
| 137 | 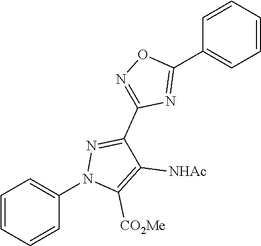 [SMILES: COC(=O)c1c(NC(C)=O)c(-c2noc(-c3ccccc3)n2)nn1-c1ccccc1] | 403.39 | 82, 31 |

1. A compound of general formula (1) 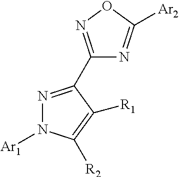
wherein
Ar₁ is unsubstituted or substituted phenyl with one to five identical or different R₃ groups selected from the group consisting of NR₅R₆, OR₅, COR₅, COOR₅, CONR₅R₆, NR₅COR₆,
—C≡CR₅ and 5-(1,2,3-triazolyl)-R₅;
Ar₂ is unsubstituted or substituted phenyl with one to five identical or different R₄ groups selected from the group consisting of Halo, NO₂, NR₅R₆, OR₅, COR₅, COOR₅, CONR₅R₆, and NR₅COR₆;
R₁ is NR₅R₆ or NR₅COR₆;
R₂ is COOR₅, CONR₅R₆ or CONR₅OR₆;
R₅ and R₆ are identical or different and are selected from H, alkyl, cycloalkyl, fluoroalkyl, phenyl, benzyl, pyridyl, CO-alkyl, CO-fluoroalkyl, CO-phenyl, CO-benzyl, NH-(cyclo)alkyl, NH-fluoroalkyl, NH-phenyl and NH-benzyl;
wherein the phenyl, benzyl and pyridyl groups are unsubstituted or substituted by one or more R₇ groups selected from the group consisting of alkyl, O-alkyl, cycloalkyl, fluoroalkyl and phenyl;
or a pharmaceutically acceptable salt thereof with the exclusion of the following compounds:
R1 is NH COCH₃, R2 is CO₂CH₃, Ar1 is 3-methoxyphenyl and Ar2 is phenyl,
R1 is NH COCH₃, R2 is CO₂CH₃, Ar1 is 3-methoxyphenyl and Ar2 is 4-methoxyphenyl and
R1 is NH COCH₃, R2 is CO₂CH₃, Ar1 is 3-methoxyphenyl and Ar2 is 4-nitrophenyl.

2. The compound of claim 1, wherein R₁ is selected from the group consisting of NH₂, NH-(cyclo) alkyl, N-((cyclo)-alkyl)₂, NHCO-(cyclo)alkyl, N(CO-(cyclo)alkyl)₂, NH-fluoroalkyl, N-(fluoroalkyl)₂, NHCO-fluoroalkyl, N—(CO-trifluoroalkyl)₂ and R₂ is selected from the group consisting of COOH, COO-alkyl, CO—NH-(cyclo)alkyl and CO—NH—O-(cyclo)alkyl.

3. The compound of claim 1, wherein:
Ar₁ is unsubstituted or substituted by one R₃ group selected from the group consisting of OR₅, COOH, COOR₅, CONHR₆ and —C≡CH, and a group of formula: 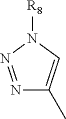
R₅ and R₆ are identical or different and are selected from the group consisting of (cyclo)alkyl, fluoroalkyl, phenyl, benzyl, pyridyl, CO-alkyl, CO-fluoroalkyl, CO-phenyl, CO-benzyl, NH-alkyl, NH-fluoroalkyl, NH-phenyl and NH-benzyl, and
R₅ is phenyl, benzyl, or pyridyl, wherein the phenyl, benzyl and pyridyl are unsubstituted or substituted by one or more of alkyl, trifluoro-alkyl and O-alkyl.

4. The compound of claim 1 of general formula II, 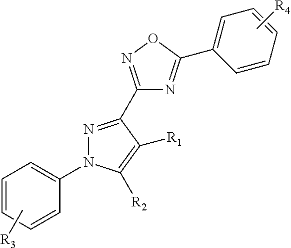
wherein
R₁ is selected from the group consisting of NH₂, NH Alkyl, N((cyclo)alkyl)₂, NHCO-(cyclo)alkyl, N (CO-(cyclo) alkyl)₂ and NH-fluoroalkyl;
R₂ is COOR₅, CONR₅R₆ or CO—NH—O alkyl;
R₃ is selected from the group consisting of H, OR₅, COOH, COOR₅, CONHR₆, —C≡CR₅, and a group of formula: 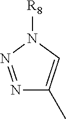
R₅ and R₆ are identical or different and are selected from the group consisting of H, (cyclo) alkyl, fluoroalkyl, phenyl, benzyl, pyridyl, CO-alkyl, CO-fluoroalkyl, CO-phenyl, CO-benzyl, NH-alkyl, NH-fluoroalkyl, NH-phenyl and NH-benzyl;
R₅ is phenyl, benzyl, or pyridyl, wherein the phenyl, benzyl and pyridyl are unsubstituted or substituted by one or more of alkyl, trifluoro-alkyl and O-alkyl;
R₄ is selected from the group consisting of H, Halo, NO₂, NH₂, NH-(cyclo) alkyl and N(-(cyclo) alkyl)₂; and
R₅ and R₆ re identical or different and are selected from the group consisting of H, alkyl, cycloalkyl, fluoroalkyl, phenyl, CO-(cyclo) alkyl, CO-fluoroalkyl and CO-phenyl;
or a pharmaceutically acceptable salt thereof with the exclusion of the following compounds:
R1 is NH COCH₃, R2 is CO₂CH₃, Ar1 is 3-methoxyphenyl and Ar2 is phenyl,
R1 is NH COCH₃, R2 is CO₂CH₃, Ar1 is 3-methoxyphenyl and Ar2 is 4-methoxyphenyl and
R1 is NH COCH₃, R2 is CO₂CH₃, Ar1 is 3-methoxyphenyl and Ar2 is 4-nitrophenyl.

5. The compound of claim 4, wherein R₃ is in the meta position and R₄ is in the ortho or para position.

6. The compound of claim 1, wherein the compound is:
Methyl-4-acetamido-3-(5-(4-bromophenyl)-1,2,4-oxadiazol-3-yl)-1-(3-ethoxyphenyl)-1H-pyrazole-5-carboxylate;
Methyl-4-acetamido-1-(3-methoxyphenyl)-3-(5-(4-nitrophenyl)-1,2,4-oxadiazol-3-yl)-1H-pyrazole-5-carboxylate;
Methyl-4-acetamido-3-(5-(4-(dimethylamino)phenyl)-1,2,4-oxadiazol-3-yl)-1-(3-methoxyphenyl)-1H-pyrazole-5-carboxylate; or
Methyl-4-acetamido-1-(3-methoxyphenyl)-3-(5-phenyl-1,2,4-oxadiazol-3-yl)-1H-pyrazole-5-carboxylate.

7-9. (canceled)

10. A pharmaceutical composition comprising a compound of claim 1 and a pharmaceutically acceptable carrier.

11. A method for treating or preventing a FGFR3-related skeletal disease comprising administering a therapeutically effective amount of at least one compound of claim 1 or a pharmaceutical composition comprising the at least one compound, to a subject in need thereof.

12. The method of claim 11, wherein the FGFR3-related skeletal disease is selected from the group consisting of thanatophoric dysplasia type I, thanatophoric dysplasia type II, severe achondroplasia with developmental delay and acanthosis nigricans, hypochondroplasia, achondroplasia and FGFR3-related craniosynostosis.

13. The method of claim 12, wherein the FGFR3-related skeletal disease is achondroplasia.

14. The method of claim 12, wherein the FGFR3-related skeletal disease is caused by expression in the subject of a constitutively active FGFR3 receptor mutant.

15. A method for treating or preventing cancer, comprising administering at least one compound of claim 1 or a composition comprising the at least one compound, to a subject in need thereof.

16. A method according to claim 15, wherein the cancer is bladder cancer.

17. The method of claim 12, wherein the FGFR3-related skeletal disease is Muenke syndrome or Crouzon syndrome with acanthosis nigricans
